SEQUENCE LISTING (24 sequences)

SEQ ID NO: 1 - protein, 10 aa, Rattus norvegicus
VFLGFTYVAP

SEQ ID NO: 2 - protein, 10 aa, Oryctolagus cuniculus
DFEGFSYVNP

SEQ ID NO: 3 - protein, 10 aa, Oryctolagus cuniculus
DFQGFTYVNP

SEQ ID NO: 4 - protein, 10 aa, Mus musculus
AFHGFSFVNP

SEQ ID NO: 5 - protein, 10 aa, Rattus norvegicus
EFKGFSYFGE

SEQ ID NO: 6 - protein, 10 aa, Xenopus laevis
LFRGFSFVAP

SEQ ID NO: 7 - protein, 10 aa, Mus musculus
LFRGFSFVAT

SEQ ID NO: 8 - protein, 10 aa, Homo sapiens
HFPQFSYSAS

SEQ ID NO: 9 - protein, 10 aa, Homo sapiens
HFPQFSYSAF

SEQ ID NO: 10 - protein, 10 aa, Drosophila melanogaster
EFAGFSFVNP

SEQ ID NO: 11 - protein, 10 aa, Caenorhabditis elegans
EFRGFSFINP

SEQ ID NO: 12 - protein, 10 aa, Arabidopsis thaliana
PFTNFTYVRP

SEQ ID NO: 13 - protein, 10 aa, Dictyostelium discoideum
SFEGFTYVAD

SEQ ID NO: 14 - protein, 10 aa, Saccharomyces cerevisiae
KFAGFTFVDE

SEQ ID NO: 15 - protein, 10 aa, Saccharomyces cerevisiae
QFGGWTYVGN

SEQ ID NO: 16 - protein, 7 aa, Artificial Sequence
  General motif (2)..(2): any amino acid residue
FXGFSYV

SEQ ID NO: 17 - protein, 7 aa, Artificial Sequence
  General motif (2)..(2): Any amino acid residue
FXGFTYV

SEQ ID NO: 18 - protein, 7 aa, Artificial Sequence
  General motif (2)..(2): any amino acid residue
FXQFSYS

SEQ ID NO: 19 - protein, 7 aa, Artificial Sequence
  General motif (2)..(2): any amino acid residue
FXGFSFV

SEQ ID NO: 20 - protein, 7 aa, Artificial Sequence
  General motif (2)..(2): any amino acid residue
FXGFSYF

SEQ ID NO: 21 - protein, 7 aa, Artificial Sequence
  General motif (2)..(2): any amino acid residue
FXGFSFI

SEQ ID NO: 22 - protein, 7 aa, Artificial Sequence
  General motif (2)..(2): any amino acid residue
FXGFTFV

SEQ ID NO: 23 - protein, 7 aa, Artificial Sequence
  General motif (2)..(2): any amino acid residue
FXGWTYV

SEQ ID NO: 24 - protein, 7 aa, Artificial Sequence
  General motif (2)..(2): any amino acid residue
FXNFTYV

(19) United States
(12) Patent Application Publication
(10) Pub. No.: US 2003/0166034 A1
(43) Pub. Date: Sep. 4, 2003

(54) P70-S6K or related kinase with constitutive activity

(76) Inventors: George Thomas, Hesingue (FR); Sara Kozma, Hesingue (FR)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/317,550

(22) Filed: Dec. 12, 2002

Related U.S. Application Data
(63) Continuation of application No. 09/297,404, filed on Apr. 29, 1999, now abandoned.
Application No. 09/297,404 is a 371 of international application No. PCT/EP97/05979, filed on Oct. 29, 1997.
(60) Provisional application No. 60/030,262, filed on Oct. 31, 1996.

(51) Int. Cl. C12Q 1/48; C12N 9/12

(52) U.S. Cl. 435/15; 435/194

(57) ABSTRACT

A protein kinase having a structure related to that of a kinase enzyme of the p70^{S6K} signalling pathway is described, wherein a serine or threonine residue homologous to T₃₈₉ in p70^{S6K} is replaced with an acidic amino acid residue in order to confer constitutive activity.

### ACTIVE KINASE

The present invention relates to an artificially activated form of a kinase related to p70^{S6K} and a method for producing it. The invention also relates to screening techniques based on such kinases.

### BACKGROUND OF THE INVENTION

Protein phosphorylation and dephosphorylation are fundamental processes for the regulation of cellular functions. They are prominently involved in signal transduction, where extracellular signals are propagated and amplified by a cascade of protein phosphorylation and dephosphorylation. Two of the best characterised signal transduction pathways involve the c-AMP-dependant protein kinase (PKA) and protein kinase C (PKC). Each pathway uses a different second messenger molecule to activate the protein kinase, which, in turn, phosphorylates specific target molecules.

Recently, a novel serine/threonine kinase has been implicated in cell growth control, termed S6 kinase or p70^{S6K}. S6 kinase phosphorylates the 40S ribosomal protein S6, an event which is believed to upregulate protein synthesis and is required in order for progression through the G₁ phase of the cell cycle. The activity of p70^{S6K} is regulated by serine/threonine phosphorylation thereof, and it is itself a serine/threonine kinase. The p70^{S6K} signalling pathway is believed to consist of a series of serine/threonine kinases, activating each other in turn and leading to a variety of effects associated with cell proliferation and growth.

Phosphorylation of p70^{S6K} has been shown to be a central event in interleukin-2 (IL-2) stimulation of T-cells. Since IL-2 stimulation leads to T-cell proliferation, compounds which inhibit such proliferation are a candidate immunosuppressive or antiproliferative therapeutic agents.

One such compound recently identified is rapamycin. In contrast to compounds such as FK-506, which inhibit cytokine gene expression and thus suppress production of IL-2 itself, the macrolide antibiotic rapamycin, which is known to be a potent immunosuppressive agent, exerts an inhibitory effect on T-cell proliferation by inhibiting the IL-2 signalling pathway at a stage downstream of the binding of IL-2 to its receptor. Rapamycin induced immunosuppression has been hypothesised to be the result of the suppression of p70^{S6K} activation and p70^{S6K} inhibitors have therefore been suggested as potential immunosuppressive and antiproliferative agents.

Other kinases are known to lie on the p70^{S6K} signalling pathway and to be responsible for successive phosphorylation events which eventually lead to the phosphorylation of p70^{S6K} and S6 itself. In particular, since p70^{S6K} is regulated by phosphorylation, the existence of a p70^{S6K} kinase has been proposed. Such a kinase kinase would be responsible for regulation of p70^{S6K} and would be a target for potential immunosuppressive and antiproliferative agents.

Moreover, still further kinases which may lie upstream on the p70^{S6K} signalling pathway are known to be able to influence p70^{S6K} activity. One such kinase, RAC (Jones, et al. (1991) Proc. Natl Acad. Sci. USA 88, 4171-4175; Jones, et al. (1991) Cell Regulation 2, 1001-1009) is known to have an activating effect on p70^{S6K}. For this reason, inhibitors of RAC and other kinases which indirectly affect the p70^{S6K} signalling pathway are candidate immunosuppressive and antiproliferative agents. Activation of RAC itself does not appear to be affected by rapamycin, which is therefore assumed to act downstream of or parallel to RAC in p70^{S6K} activation.

When isolated from natural sources, especially convenient sources such as tissue culture cells, RAC, p70^{S6K} and other related kinases are normally in the inactive state. In order to isolate active kinase proteins, it is necessary to stimulate cells in order to switch on the appropriate signalling pathway. Thus, cells are normally stimulated with mitogens and/or agents such as IL-2, platelet-derived growth factor (PDGF), insulin, epidermal growth factor (EGF) and basic fibroblast growth factor (bFGF). Such agents are expensive and, when it is desired to produce active kinases in large amounts, the use of such agents is disadvantageous.

Screening of candidate compounds for activity as inhibitors of RAC, p70^{S6K} or other kinases on the p70^{S6K} pathway in order to identify candidate immunosuppressive or antiproliferative agents requires plentiful supplies of kinase protein. Using recombinant DNA technology, it is possible to produce large quantities of virtually any desired protein in heterologous expression systems. In the case of kinases such as those with which we are presently concerned, however, such systems are unsatisfactory because the proteins produced would be unphosphorylated and therefore inactive. There is therefore a requirement to identify a cost-effective way to produce phosphorylated kinase proteins which can be employed in screening procedures.

It is known that activation of kinases of the p70^{S6K} signalling pathway is dependent upon phosphorylation at certain sites, and moreover that phosphorylation of proteins in general may in certain cases be mimicked by replacement of the phosphorylated amino acid (usually serine or threonine) with an acidic amino acid, such as aspartic acid or glutamic acid. By examining the phosphorylation sites of active p70^{S6K} we have been able to determine which phosphorylation sites are important targets for inactivation by rapamycin and which sites are responsible for kinase activation. The major rapamycin-sensitive phosphorylation site of p70^{S6K}, which by implication is essential for kinase activation, is found to be conserved throughout p70^{S6K} related kinases. Replacement of the phosphorylated residue with an acidic amino acid, such as aspartic acid, produces a constitutively active kinase which can be produced in heterologous expression systems and yet retain its activity.

### SUMMARY OF THE INVENTION

According to the invention, we provide a kinase enzyme having a structure related to that of a kinase enzyme of the p70^{S6K} signalling pathway wherein a serine or threonine residue homologous to T₃₈₉ in p70^{S6K} is replaced with an acidic amino acid in order to confer constitutive activity. Moreover, we provide screening systems which monitor for dephosphorylation of the major rapamycin sensitive sites in p70^{S6K}-related kinases and/or use constitutively active or inactive mutant kinases modified at these sites to identify further kinases on the p70^{S6K} pathway and agents which modulate this pathway.

### DETAILED DESCRIPTION OF THE INVENTION

The p70^{S6K} signalling pathway is known to comprise a number of sequential phosphorylation steps catalysed by a series of serine/threonine kinases. Those kinases of the p70^{S6K} pathway which have been characterised, as well as related kinases including homologous kinases from other species, contain a conserved serine or threonine residue homologous to p70^{S6K} T₃₈₉ which may be clearly identified by sequence alignment, as shown below. For the avoidance of doubt, p85^{S6K}, the nuclear form of p70^{S6K}, is considered equivalent to p70^{S6K} for the purposes of this invention.

This residue has been found to be a major target for rapamycin-induced dephosphorylation. Substitution of this residue with a neutral residue such as alanine results in loss of basal kinase activity and loss of susceptibility to activation. It can be deduced that T₃₈₉ is a crucial residue in p70^{S6K} activation. In accordance with this observation, we have found that replacement of T₃₈₉ with an acidic amino acid, preferably glutamic acid (E) or possibly aspartic acid (D) results in a several-fold increase in basal kinase activity together with a retention of susceptibility to activation. It will be appreciated, therefore, that constitutively active kinases according to the invention do not necessarily possess 100% constitutive activity, but may be susceptible to activation; their basal activity in the absence of activation, however, is substantially increased with respect to the wild-type kinase.

By “related” it is intended to denote that, in the case of a kinase enzyme, the enzyme in question has a similar structure to an enzyme from the p70^{S6K} signalling pathway in the context of activation and inactivation by phosphorylation. Thus, a homologous enzyme possesses similar domains arranged in a like manner and performs a similar function in its native context, although it is not to be excluded that additional domains will be present or some may be omitted. Most importantly, however, related kinases share similarity of structure in regions concerned with activation thereof. A non-exhaustive list of related kinases in accordance with the present invention is set forth below.

In the case of a “homologous” amino acid residue, it is intended to indicate that the homologous amino acids are present in a similar positional context and perform a similar role in different but related enzymes. For example, the residue homologous to T₃₈₉ in kinases related to p70^{S6K} is located within a conserved sequence motif which has the structure F-GFT/SYV. In certain cases, the structure of this motif may vary. RAC kinase, for example, has the motif F-QFSYS. Other amino acid substitutions may occur, as follows:

| | | |
|---|---|---|
| p70^{S6K} | V F L G F T Y V A P | SEQ ID No. 1 |
| PKCα | D F E G F S Y V N P | SEQ ID No. 2 |
| PKCγ | D F Q G F T Y V N P | SEQ ID No. 3 |
| PKCδ | A F H G F S F V N P | SEQ ID No. 4 |
| PKCε | E F K G F S Y F G E | SEQ ID No. 5 |
| RSK xen1 | L F R G F S F V A P | SEQ ID No. 6 |
| RSK mα1 | L F R G F S F V A T | SEQ ID No. 7 |
| RAC-PK/Akt α | H F P Q F S Y S A S | SEQ ID No. 8 |
| RAC-PK/Akt β | H F P Q F S Y S A F | SEQ ID No. 9 |
| KPC3 drome | E F A G F S F V N P | SEQ ID No. 10 |
| KPC2 caeel | E F R G F S F I N P | SEQ ID No. 11 |
| ATPK1 | P F T N F T Y V R P | SEQ ID No. 12 |
| PK2 dicdi | S F E G F T Y V A D | SEQ ID No. 13 |
| SCH9 | K F A G F T F V D E | SEQ ID No. 14 |
| YPK1 | Q F G G W T Y V G N | SEQ ID No. 15 |

Preferably, therefore, the T₃₈₉ equivalent mutation in an p70^{S6K}-related kinase is comprised in a motif homologous to F-GFT/SYV, for example one identified in the above list. What will be apparent, however, is that the T₃₈₉-homologous amino acid is in each case flanked by large aromatic groups, usually flanked by glycine at −2, valine at +2 and proline at +4, with an invariant phenylalanine at −4. Moreover, the homology extends throughout all PKC isoforms, p70^{S6K}, RAC and equivalents from lower organisms such as moulds and yeast.

Preferably, the T₃₈₉ mutation is combined with one or more additional serine or threonine substitutions involving insertion of an acidic amino acid in place thereof. For example, combination of the T₃₈₉->E with one or more of the D₃E mutations is particularly advantageous.

The D₃E mutations as herein defined comprises the mutations previously described in p70^{S6K} by Ferrari et al., (1993) J. Biol. Chem. 268, 16091-16094, and equivalent mutations in related kinases. These mutations consist in the conversion of S₄₁₁, S₄₁₈ and S₄₂₄ to D and the conversion of T₄₂₁ to E. The combination of any one or more of these mutations with the T₃₈₉->E mutation further increases the basal activity of p70^{S6K} and imparts rapamycin resistance thereto. Preferably, the T₃₈₉->E mutation is combined with all of the D₃E mutations.

Moreover, mutations may be effected at sites homologous to S₄₀₄ on p70^{S6K}. In general, threonine residues are converted to glutamic acid and serine residues are converted to aspartic acid.

Interestingly, although T₃₈₉ is the major rapamycin-sensitive site involved in the regulation of p70^{S6K}, it is not the only site involved in p70^{S6K} activation. A second site which must be phosphorylated for p70^{S6K} activation is T₂₂₉. Dephosphorylation of this site results in immediate loss of any kinase activity. Moreover, mutation of this site to an acidic amino acid results in irreversible loss of kinase activity. Phosphorylation of T₂₂₉ is itself dependent on phosphorylation of T₃₈₉. Thus, a regulatory p70^{S6K} kinase acts on T₃₈₉, phosphorylation of this site being responsible for activation of a further p70^{S6K} kinase which phosphorylates T₂₂₉.

The invention accordingly provides a kinase enzyme having a structure related to that of a kinase enzyme of the p70^{S6K} signalling pathway comprising the D₃E and T₃₈₉->E mutation. Such a kinase enzyme is useful as a tool for identifying p70^{S6K} kinases responsible for both regulation of the p70^{S6K} related kinase and direct activation thereof through T₂₂₉.

Moreover, the invention provides provide a kinase enzyme having a structure related to that of a kinase enzyme of the p70^{S6K} signalling pathway comprising a T₂₂₉->A and/or a K₁₀₀->E mutation. These mutants are dominant negative mutants of p70^{S6K}, irreversibly inactive and capable of preventing activation of endogenous p70^{S6K} in a cell by competing therewith for factors essential for p70^{S6K} activation. The K₁₀₀ residue lies in the ATP binding site of p70^{S6K}.

The dominant negative mutants compete for the upstream kinase kinase enzyme responsible for p70^{S6K} activation and prevent its becoming available to phosphorylate endogenous p70^{S6K} and other downstream targets. The mutants are useful as tools for defining the p70^{S6K} signalling pathway, and as agents for blocking the upstream kinase kinase.

Mutations may be performed by any method known to those of skill in the art. Preferred, however, is site-directed mutagenesis of a nucleic acid sequence encoding the kinase of interest. A number of methods for site-directed mutagenesis are known in the art, from methods employing single-stranded phage such as M13 to PCR-based techniques (see “PCR Protocols: A guide to methods and applications”, M. A. Innis, D. H. Gelfand, J. J. Sninsky, T. J. White (eds.). Academic Press, New York, 1990). Preferably, the commercially available Altered Site II Mutagenesis System (Promega) may be employed, according to the directions given by the manufacturer.

Nucleic acids encoding a variety of p70^{S6K}-related kinases are known to those of skill in the art and may be obtained readily by de novo cloning (see below), from academic sources, depository institutions or by automated synthesis of published sequence information. For example, p70^{S6K}-related kinases and nucleic aids encoding them are currently described in:

| | |
|---|---|
| p70^{S6K} | Kozma, S. C., et al., (1990) Proc. Natl. Acad. Sci. |
| | USA 87, 7365-7369. |
| PKCα | Ohno, S., et al., (1988) Biochemistry 27, 2083-2087. |
| PKCγ | Ohno, S., et al., (1988) Biochemistry 27, 2083-2087. |
| PKCδ | Mischak, H., et al., (1991) Biochemistry 30, 7925-7931. |
| PKCε | Ono Y. et al., (1988) J. Biol. Chem., 263, 6927-6932. |
| RSK xen1 | Jones, S. W., et al., (1988) Proc. Natl. Acad. Sci. |
| | USA 85, 3377-3381. |
| RSK mα1 | Alcorta, D.A., et al., (1989) Mol. Cell. Biol., 9, |
| | 3850-3859. |
| RAC-PK/Akt α | Jones, P. F., et al., (1991) Proc. Natl. Acad. Sci. |
| | USA, 88, 4171-4175. |
| RAC-PK/Akt β | Jones, P. F. et al., (1991) Cell Regul., 2, 1001-1009. |
| KPC3 drome | Schaeffer, E., et al., (1989) Cell, 57, 403-412. |
| KPC2 caeel | Land, M., et al., (1994) J. Biol. Chem., 269, 9234-9244. |
| ATPK1 | Zhang, S. -H., et al., (1994) J. Biol. Chem. 269, |
| | 17586-17592. |
| PK2 dicdi | Haribabu, B. et al., (1991) Proc. Natl. Acad. Sci. |
| | USA, 88, 1115-1119. |
| SCH9 | Toda, T., et al., (1988) Genes & Dev., 2, 517-527. |
| YPK1 | Maurer, R. A. (1988) DNA 7, 469-474. |

If required, nucleic acids encoding p70^{S6K}-related kinases may be cloned from tissues according to established procedures using probes derived from p70^{S6K} itself or any of the published sequences of related kinases. In particular, such DNAs can be prepared by:

a) isolating mRNA from suitable cells, for example human embryonic kidney 293 cells or Swiss 3T3 cells, selecting the desired mRNA, for example by hybridisation with a DNA probe or by expression in a suitable expression system and screening for expression of the desired polypeptide, preparing single-stranded cDNA complementary to that mRNA, then double-stranded cDNA therefrom, or

b) isolating cDNA from a cDNA library and selecting the desired cDNA, for example using a DNA probe or using a suitable expression system and screening for expression of the desired polypeptide, or

c) incorporating the double-stranded DNA of step a) or b) into an appropriate expression vector,

d) transforming appropriate host cells with the vector and isolating the desired DNA.

Polyadenylated messenger RNA (step a) is isolated by known methods. Isolation methods involve, for example, homogenizing cells in the presence of a detergent and a ribonuclease inhibitor, for example heparin, guanidinium isothiocyanate or mercaptoethanol, extracting the mRNA with a chloroform-phenol mixture, optionally in the presence of salt and buffer solutions, detergents and/or cation chelating agents, and precipitating mRNA from the remaining aqueous, salt-containing phase with ethanol, isopropanol or the like. The isolated mRNA may be further purified by centrifuging in a caesium chloride gradient followed by ethanol precipitation and/or by chromatographic methods, for example affinity chromatography, for example chromatography on oligo(dt) cellulose or on oligo(U) sepharose. Preferably, such purified total mRNA is fractionated according to size by gradient centrifugation, for example in a linear sucrose gradient, or chromatography on suitable size fractionation columns, for example on agarose gels.

The desired mRNA is selected by screening the mRNA directly with a DNA probe, or by translation in suitable cells or cell-free systems and screening the obtained polypeptides.

The selection of the desired mRNA is preferably achieved using a DNA hybridisation probe, thereby avoiding the additional step of translation. Suitable DNA probes are DNAs of known nucleotide sequence consisting of at least 17 nucleotides derived from DNAs encoding p70^{S6K} or a related kinase.

Synthetic DNA probes are synthesised according to known methods as detailed hereinbelow, preferably by stepwise condensation using the solid phase phosphotriester, phosphite triester or phosphoramidite method, for example the condensation of dinucleotide coupling units by the phosphotriester method. These methods are adapted to the synthesis of mixtures of the desired oligonucleotides by using mixtures of two, three or four nucleotides dA, dC, dG and/or dT in protected form or the corresponding dinucleotide coupling units in the appropriate condensation step as described by Y. Ike et al. (Nucleic Acids Research 11, 477, 1983).

For hybridisation, the DNA probes are labelled, for example radioactively labelled by the well known kinase reaction. The hybridisation of the size-fractionated mRNA with the DNA probes containing a label is performed according to known procedures, i.e. in buffer and salt solutions containing adjuncts, for example calcium chelators, viscosity regulating compounds, proteins, irrelevant DNA and the like, at temperatures favouring selective hybridisation, for example between 0° C. and 80° C., for example between 25° C. and 50° C. or around 65° C., preferably at around 200 lower than the hybrid double-stranded DNA melting temperature.

Fractionated mRNA may be translated in cells, for example frog oocytes, or in cell-free systems, for example in reticulocyte lysates or wheat germ extracts. The obtained polypeptides are screened for kinase activity or for reaction with antibodies raised against the p70^{S6K} related kinase, for example in an immunoassay, for example radioimmunoassay, enzyme immunoassay or immunoassay with fluorescent markers. Such immunoassays and the preparation of polyclonal and monoclonal antibodies are well known in the art and are applied accordingly.

The preparation of a single-stranded complementary DNA (cDNA) from the selected mRNA template is well known in the art, as is the preparation of a double-stranded DNA from a single-stranded DNA. The mRNA template is incubated with a mixture of deoxynucleoside triphosphates, optionally radioactively labelled deoxynucleoside triphosphates (in order to be able to screen the result of the reaction), a primer sequence such as an oligo-dT residue hybridising with the poly(A) tail of the mRNA and a suitable enzyme such as a reverse transcriptase for example from avian myeloblastosis virus (AMV). After degradation of the template mRNA for example by alkaline hydrolysis, the cDNA is incubated with a mixture of deoxynucleoside triphosphates and a suitable enzyme to give a double-stranded DNA. Suitable enzymes are for instance a reverse transcriptase, the Klenow fragment of E. coli DNA polymerase I or T4 DNA polymerase. Usually, a hairpin loop structure formed spontaneously by the single-stranded cDNA acts as a primer for the synthesis of the second strand. This hairpin structure is removed by digestion with S1 nuclease. Alternatively, the 3′-end of the single-stranded DNA is first extended by homopolymeric deoxynucleotide tails prior to the hydrolysis of the mRNA template and the subsequent synthesis of the second cDNA strand.

In the alternative, double-stranded cDNA is isolated from a cDNA library and screened for the desired cDNA (step b). The cDNA library is constructed by isolating mRNA from suitable cells, for example human mononuclear leukocytes or human embryonic epithelial lung cells, and preparing single-stranded and double-stranded cDNA therefrom as described above. This cDNA is digested with suitable restriction endonucleases and incorporated into λ phage, for example λ charon 4A or λ gt11 following established procedures. The cDNA library replicated on nitrocellulose membranes is screened by using a DNA probe as described hereinbefore, or expressed in a suitable expression system and the obtained polypeptides screened for reaction with an antibody specific for the desired kinase, for example an antibody specific for p70^{S6K}.

A variety of methods are known in the art for the incorporation of double-stranded cDNA into an appropriate vector (step c). For example, complementary homopolymer tracts may be added to the double-stranded DNA and the vector DNA by incubation in the presence of the corresponding deoxynucleoside triphosphates and an enzyme such as terminal deoxynucleotidyl transferase. The vector and double-stranded DNA are then joined by base pairing between the complementary homopolymeric tails and finally ligated by specific joining enzymes such as ligases. Other possibilities are the addition of synthetic linkers to the termini of the double-stranded DNA, or the incorporation of the double-stranded DNA into the vector by blunt- or staggered-end ligation.

The transformation of appropriate host cells with the obtained hybrid vector (step d) and the selection of transformed host cells (step e) are well known in the art. Hybrid vectors and host cells may be particularly suitable for the production of DNA, or for the production of the desired kinase.

The isolation of the desired DNA is achieved by methods known in the art, for example extraction with phenol and/or chloroform or glass beads. Optionally, the DNA can be further manipulated for example by treatment with mutagenic agents to obtain mutants, or by digestion with restriction enzymes to obtain fragments, modify one or both termini to facilitate incorporation into the vector.

Once a kinase-encoding nucleic acid as set forth above has been mutated according to the invention, it may be expressed in any one of a variety of expression systems which are available to a person of skill in the art. Such systems include prokaryotic, eukaryotic, insect and fungal expression systems, and are described below.

The cDNA or genomic DNA encoding native or mutant p70^{S6K} can be incorporated into vectors for further manipulation. As used herein, vector (or plasmid) refers to discrete elements that are used to introduce heterologous DNA into cells for either expression or replication thereof. Selection and use of such vehicles are well within the skill of the artisan. Many vectors are available, and selection of appropriate vector will depend on the intended use of the vector, i.e. whether it is to be used for DNA amplification or for DNA expression, the size of the DNA to be inserted into the vector, and the host cell to be transformed with the vector. Each vector contains various components depending on its function (amplification of DNA or expression of DNA) and the host cell for which it is compatible. The vector components generally include, but are not limited to, one or more of the following: an origin of replication, one or more marker genes, an enhancer element, a promoter, a transcription termination sequence and a signal sequence.

Both expression and cloning vectors generally contain nucleic acid sequence that enable the vector to replicate in one or more selected host cells. Typically in cloning vectors, this sequence is one that enables the vector to replicate independently of the host chromosomal DNA, and includes origins of replication or autonomously replicating sequences. Such sequences are well known for a variety of bacteria, yeast and viruses. The origin of replication from the plasmid pBR322 is suitable for most Gram-negative bacteria, the 2μ plasmid origin is suitable for yeast, and various viral origins (e.g. SV 40, polyoma, adenovirus) are useful for cloning vectors in mammalian cells. Generally, the origin of replication component is not needed for mammalian expression vectors unless these are used in mammalian cells competent for high level DNA replication, such as COS cells.

Most expression vectors are shuttle vectors, i.e. they are capable of replication in at least one class of organisms but can be transfected into another organism for expression. For example, a vector is cloned in E. coli and then the same vector is transfected into yeast or mammalian cells even though it is not capable of replicating independently of the host cell chromosome. DNA may also be replicated by insertion into the host genome. However, the recovery of genomic DNA encoding p70^{S6K} related kinase is more complex than that of exogenously replicated vector because restriction enzyme digestion is required to excise p70^{S6K} related kinase DNA. DNA can be amplified by PCR and be directly transfected into the host cells without any replication component.

Advantageously, an expression and cloning vector may contain a selection gene also referred to as selectable marker. This gene encodes a protein necessary for the survival or growth of transformed host cells grown in a selective culture medium. Host cells not transformed with the vector containing the selection gene will not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available from complex media.

As to a selective gene marker appropriate for yeast, any marker gene can be used which facilitates the selection for transformants due to the phenotypic expression of the marker gene. Suitable markers for yeast are, for example, those conferring resistance to antibiotics G418, hygromycin or bleomycin, or provide for prototrophy in an auxotrophic yeast mutant, for example the URA3, LEU2, LYS2, TRP1, or HIS3 gene.

Since the replication of vectors is conveniently done in E. coli, an E. coli genetic marker and an E. coli origin of replication are advantageously included. These can be obtained from E. coli plasmids, such as pBR322, Bluescript® vector or a pUC plasmid, e.g. pUC18 or pUC19, which contain both E. coli replication origin and E. coli genetic marker conferring resistance to antibiotics, such as ampicillin.

Suitable selectable markers for mammalian cells are those that enable the identification of cells competent to take up p70^{S6K} related kinase nucleic acid, such as dihydrofolate reductase (DHFR, methotrexate resistance), thymidine kinase, or genes conferring resistance to G418 or hygromycin. The mammalian cell transformants are placed under selection pressure which only those transformants which have taken up and are expressing the marker are uniquely adapted to survive. In the case of a DHFR or glutamine synthase (GS) marker, selection pressure can be imposed by culturing the transformants under conditions in which the pressure is progressively increased, thereby leading to amplification (at its chromosomal integration site) of both the selection gene and the linked DNA that encodes s6k. Amplification is the process by which genes in greater demand for the production of a protein critical for growth, together with closely associated genes which may encode a desired protein, are reiterated in tandem within the chromosomes of recombinant cells. Increased quantities of desired protein are usually synthesised from thus amplified DNA.

Expression and cloning vectors usually contain a promoter that is recognised by the host organism and is operably linked to p70^{S6K} related kinase nucleic acid. Such a promoter may be inducible or constitutive. The promoters are operably linked to DNA encoding p70^{S6K} related kinase by removing the promoter from the source DNA by restriction enzyme digestion and inserting the isolated promoter sequence into the vector. Both the native p70^{S6K} related kinase promoter sequence and many heterologous promoters may be used to direct amplification and/or expression of p70^{S6K} related kinase DNA.

Promoters suitable for use with prokaryotic hosts include, for example, the β-lactamase and lactose promoter systems, alkaline phosphatase, the tryptophan (trp) promoter system and hybrid promoters such as the tac promoter. Their nucleotide sequences have been published, thereby enabling the skilled worker operably to ligate them to DNA encoding p70^{S6K} related kinase, using linkers or adaptors to supply any required restriction sites. Promoters for use in bacterial systems will also generally contain a Shine-Delgarno sequence operably linked to the DNA encoding p70^{S6K} related kinase.

Moreover, the p70^{S6K} related kinase gene according to the invention preferably includes a secretion sequence in order to facilitate secretion of the polypeptide from bacterial hosts, such that it will be produced as a soluble native peptide rather than in an inclusion body. The peptide may be recovered from the bacterial periplasmic space, or the culture medium, as appropriate.

Suitable promoting sequences for use with yeast hosts may be regulated or constitutive and are preferably derived from a highly expressed yeast gene, especially a Saccharomyces cerevisiae gene. Thus, the promoter of the TRP1 gene, the ADHI or ADHII gene, the acid phosphatase (PH05) gene, a promoter of the yeast mating pheromone genes coding for the α- or a-factor or a promoter derived from a gene encoding a glycolytic enzyme such as the promoter of the enolase, glyceraldehyde-3-phosphate dehydrogenase (GAP), 3-phospho glycerate kinase (PGK), hexokinase, pyruvate decarboxylase, phosphofructokinase, glucose-6-phosphate isomerase, 3-phosphoglycerate mutase, pyruvate kinase, triose phosphate isomerase, phosphoglucose isomerase or glucokinase genes, or a promoter from the TATA binding protein (TBP) gene can be used. Furthermore, it is possible to use hybrid promoters comprising upstream activation sequences (UAS) of one yeast gene and downstream promoter elements including a functional TATA box of another yeast gene, for example a hybrid promoter including the UAS(s) of the yeast PH05 gene and downstream promoter elements including a functional TATA box of the yeast GAP gene (PH05-GAP hybrid promoter). A suitable constitutive PHO5 promoter is e.g. a shortened acid phosphatase PH05 promoter devoid of the upstream regulatory elements (UAS) such as the PH05 (−173) promoter element starting at nucleotide −173 and ending at nucleotide −9 of the PH05 gene.

p70^{S6K} related kinase gene transcription from vectors in mammalian hosts may be controlled by promoters derived from the genomes of viruses such as polyoma virus, adenovirus, fowlpox virus, bovine papilloma virus, avian sarcoma virus, cytomegalovirus (CMV), a retrovirus and Simian Virus 40 (SV40), from heterologous mammalian promoters such as the actin promoter or a tissue specific promoter, and from the promoter normally associated with p70^{S6K} related kinase sequence, provided such promoters are compatible with the host cell systems.

Transcription of a DNA encoding p70^{S6K} related kinase by higher eukaryotes may be increased by inserting an enhancer sequence into the vector. Enhancers are relatively orientation and position independent. Many enhancer sequences are known from mammalian genes (e.g. elastase and globin). However, typically one will employ an enhancer from a eukaryotic cell virus. Examples include the SV40 enhancer on the late side of the replication origin (bp 100-270) and the CMV early promoter enhancer. The enhancer may be spliced into the vector at a position 5′ or 3′ to p70^{S6K} related kinase DNA, but is preferably located at a site 5′ from the promoter.

Advantageously, a eukaryotic expression vector encoding p70^{S6K} related kinase may comprise a locus control region (LCR). LCRs are capable of directing high-level integration site independent expression of transgenes integrated into host cell chromatin, which is of importance especially where the p70^{S6K} related kinase gene is to be expressed in the context of a permanently-transfected eukaryotic cell line in which chromosomal integration of the vector has occurred, in vectors designed for gene therapy applications or in transgenic animals.

Suitable eukaryotic host cells for expression of p70^{S6K} related kinase include yeast, fungi, insect, plant, animal, human, or nucleated cells from other multicellular organisms will also contain sequences necessary for the termination of transcription and for stabilising the mRNA. Such sequences are commonly available from the 5′ and 3′ untranslated regions of eukaryotic or viral DNAs or cDNAs. These regions contain nucleotide segments transcribed as polyadenylated fragments in the untranslated portion of the mRNA encoding p70^{S6K} related kinase.

An expression vector includes any vector capable of expressing p70^{S6K} related kinase nucleic acids that are operatively linked with regulatory sequences, such as promoter regions, that are capable of expression of such DNAs. Thus, an expression vector refers to a recombinant DNA or RNA construct, such as a plasmid, a phage, recombinant virus or other vector, that upon introduction into an appropriate host cell, results in expression of the cloned DNA. Appropriate expression vectors are well known to those with ordinary skill in the art and include those that are replicable in eukaryotic and/or prokaryotic cells and those that remain episomal or those which integrate into the host cell genome. For example, DNAs encoding p70^{S6K} related kinase may be inserted into a vector suitable for expression of cDNAs in mammalian cells, e.g. a CMV enhancer-based vector such as pEVRF (Matthias, et al., (1989) NAR 17, 6418).

Particularly useful for practising the present invention are expression vectors that provide for the transient expression of DNA encoding p70^{S6K} related kinase in mammalian cells. Transient expression usually involves the use of an expression vector that is able to replicate efficiently in a host cell, such that the host cell accumulates many copies of the expression vector, and, in turn, synthesises high levels of p70^{S6K} related kinase. For the purposes of the present invention, transient expression systems are useful e.g. for identifying p70^{S6K} related kinase mutants, to identify potential phosphorylation sites, or to characterise functional domains of the protein.

Construction of vectors according to the invention employs conventional ligation techniques. Isolated plasmids or DNA fragments are cleaved, tailored, and religated in the form desired to generate the plasmids required. If desired, analysis to confirm correct sequences in the constructed plasmids is performed in a known fashion. Suitable methods for constructing expression vectors, preparing in vitro transcripts, introducing DNA into host cells, and performing analyses for assessing p70^{S6K} related kinase expression and function are known to those skilled in the art. Gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe based on a sequence provided herein. Those skilled in the art will readily envisage how these methods may be modified, if desired.

Baculovirus vectors, specifically intended for insect cell culture, are widely obtainable commercially (e.g. from Invitrogen and Clontech). Other virus vectors capable of infecting insect cells are known, such as Sindbis virus (Hahn et al., (1992) PNAS (USA) 89, 2679-2683). The baculovirus vector of choice (reviewed by Miller (1988) Ann. Rev. Microbiol. 42, 177-199) is Autographa californica multiple nuclear polyhedrosis virus, AcMNPV.

Molecular biology techniques useful for practising the present invention are entirely conventional and well known to those of ordinary skill in the art. They are reviewed generally in Sambrook, (1989) Molecular Cloning; A Laboratory Manual, Cold Spring Harbor, N.Y., USA. Baculovirus techniques are equally standard and well known in the art, and are reviewed in O'Reilly et al., (1994) Baculovirus expression vectors; A laboratory manual, Oxford University Press Inc., NY, USA, as well as in literature published by suppliers of commercial baculovirus kits (e.g. Pharmingen).

Culture media suitable for insect cell culture are known in the art and typically contain 5-10% fetal calf serum (FCS). However, culture media which are serum-free are known (Ingley et al., (1991) Eur. J. Biochem. 196, 623-629; DiSorbo et al., (1991) Focus 13, 16-18) and are preferred for use in the present invention. The use of entirely protein-free medium is also possible.

Insect cells suitable for use in the method of the invention include, in principle, any lepidopteran cell which is capable of being transformed with an expression vector and expressing heterologous proteins encoded thereby. In particular, use of the Sf cell lines, such as the Spodoptera frugiperda cell line IPBL-SF-21 AE (Vaughn et al., (1977) In Vitro, 13, 213-217) is preferred. The derivative cell line Sf9 is particularly preferred. However, other cell lines, such as Tricoplusia ni 368 (Kurstack and Marmorosch, (1976) Invertebrate Tissue Culture Applications in Medicine, Biology and Agriculture. Academic Press, New York, USA) may be employed. These cell lines, as well as other insect cell lines suitable for use in the invention, are commercially available (e.g. from Stratagene, La Jolla, Calif., USA).

A preferred method for expressing kinases according to the invention comprises expression thereof in eukaryotic cell lines, especially human 293 cells. In accordance with another embodiment of the present invention, therefore, there are provided cells containing the above-described nucleic acids. Hosts suitable for p70^{S6K} related kinase encoding vectors include eukaryotic microbes such as filamentous fungi or yeast, e.g. Saccharomyces cerevisiae. Higher eukaryotic cells include insect and vertebrate cells, particularly mammalian cells. In recent years propagation of vertebrate cells in culture (tissue culture) has become a routine procedure. Examples of useful mammalian host cell lines are epithelial or fibroblastic cell lines such as Chinese hamster ovary (CHO) cells, NIH 3T3 cells, HeLa cells or 293T cells. 293T cells are especially preferred

DNA may be stably incorporated into cells or may be transiently expressed using methods known in the art. Stably transfected mammalian cells may be prepared by transfecting cells with an expression vector having a selectable marker gene, and growing the transfected cells under conditions selective for cells expressing the marker gene. To prepare transient transfectants, mammalian cells are transfected with a reporter gene to monitor transfection efficiency.

To produce such stably or transiently transfected cells, the cells should be transfected with a sufficient amount of p70^{S6K} related kinase-encoding nucleic acid to form p70^{S6K} related kinase. The precise amounts of DNA encoding p70^{S6K} related kinase may be empirically determined and optimised for a particular cell and assay.

Host cells are transfected or, preferably, transformed with the above-captioned expression or cloning vectors of this invention and cultured in conventional nutrient media modified as appropriate for inducing promoters, selecting transformants, or amplifying the genes encoding the desired sequences. Heterologous DNA may be introduced into host cells by any method known in the art, such as transfection with a vector encoding a heterologous DNA by the calcium phosphate coprecipitation technique or by electroporation. Numerous methods of transfection are known to the skilled worker in the field. Successful transfection is generally recognised when any indication of the operation of this vector occurs in the host cell. Transformation is achieved using standard techniques appropriate to the particular host cells used.

Incorporation of cloned DNA into a suitable expression vector, transfection of eukaryotic cells with a plasmid vector or a combination of plasmid vectors, each encoding one or more distinct genes or with linear DNA, and selection of transfected cells are well known in the art (see, e.g. Sambrook et al. (1989) Molecular Cloning: A Laboratory Manual, Second Edition, Cold Spring Harbor Laboratory Press).

Transfected or transformed cells are cultured using media and culturing methods known in the art, preferably under conditions, whereby p70^{S6K} related kinase encoded by the DNA is expressed. The composition of suitable media is known to those in the art, so that they can be readily prepared. Suitable culturing media are also commercially available.

According to a second aspect of the present invention, we provide a method for screening for antiproliferative or immunosuppressive agents which inhibit p70^{S6K}-related kinases, as defined hereinbefore, comprising assaying for dephosphorylation of the T₃₈₉-equivalent site in response to the inhibitor. This may be carried out, for example, by tryptic phosphopeptide mapping to monitor the disappearance of the T₃₈₉ equivalent site during time-course treatment with a putative inhibitory agent (see Ferrari, S. et al., (1992) Proc. Natl. Acad. Sci. USA 89, 7282-7285).

Thus, a method according to the invention preferably comprises the following steps:

(a) exposing a p70^{S6K}-related kinase to a candidate antiproliferative or immunosuppressive agent, in the presence of a mitogen or other activator of the kinase; and (b) assessing the presence of phosphorylation on the T₃₈₉ homologous site during the course of the exposure.

Agents identified as potential kinase inhibitors in this manner may additionally be assayed for additional activity against other phosphorylation sites, such as the D₃E sites as defined hereinbefore.

Agents may be further assessed by assay of the biological activity of a p70^{S6K}-related kinase by including a substrate for the kinase in the assay mixture and assessing the state of phosphorylation of the substrate, inhibition of phosphorylation being an indication of kinase inhibition. Moreover, kinase activity can be monitored by isolating the kinase and assessing its activity in isolated form according to known techniques, for example as described by Lane and Thomas, (1991) Meth. Enzymol., 200, 269-291.

Kinase enzyme useful in the assays described herein may be isolated, either from transfected or normal cells, by standard isolation procedures, for example as generally set forth in Deutscher (1990) Guide to Protein Purification, Academic Press, Inc., San Diego, Calif., USA, 317-328. For example, kinase may be isolated by cation exchange chromatography followed by affinity chromatography, for example using ATP-substituted agarose (see Jenö, et al., (1989) J. Biol. Chem., 264, 1293-1297). Alternatively, the kinase may be isolated by immuno-affinity chromatography.

An alternative method for isolating the kinase is by immunoprecipitation from cell lysates. Antibodies suitable for immunoprecipitation or immuno-affinity chromatography may be prepared according to conventional techniques, known to those of ordinary skill in the art, and may be monoclonal or polyclonal in nature. For example, see Lane, et al., (1992) EMBO J., 11, 1743-1749.

Preferably, a kinase according to the invention may be isolated by exploiting the presence of a tag. Thus, the kinase according to the invention may be expressed as a GST fusion, with GST being preferably located at the C terminus of the kinase enzyme. GST fusion proteins are easily isolatable by affinity chromatography acceding to known techniques.

According to a third aspect of the invention, we provide a method for screening for inhibitors of the p70^{S6K} signalling pathway comprising assaying for inhibitors of a constitutively activated p70^{S6K}-related kinase enzyme as hereinbefore described.

Use of a constitutively activated enzyme allows screening of the effect of an agent on the p70^{S6K} signalling pathway at sites below that of the activated kinase. Thus, for example, using a constitutively active p70^{S6K} it is possible to screen for agents which inhibit the activity thereof by assaying the phosphorylation state of S6 itself. Alternatively, using a constitutively active RAC kinase, which is placed upstream of p70^{S6K} and, indirectly, activates p70^{S6K}, it is possible to screen for agents which inactivate RAC or another effector which lies between RAC and p70^{S6K} by monitoring p70^{S6K} phosphorylation and activity.

The advantage of using a constitutively active kinase is that the signalling pathway may be maintained in the active state without the intervention of exogenous mitogens or other activators, such as IL-2, and the expense associated therewith.

The method of the third aspect of the invention, therefore, includes the steps of:

(a) combining a constitutively active kinase according to the first aspect of the present invention with a direct or indirect substrate therefor;

(b) exposing the combination to a candidate immunosuppressive or antiproliferative agent; and

(c) assessing the phosphorylation state of the substrate, lack of phosphorylation being indicative of inhibition of the kinase by the agent.

The assay may be conducted in cell culture as well as in solution. Preferably, cells, such as Swiss 3T3 or human embryonic kidney 293 cells, are transfected with a gene encoding a constitutively activated kinase. Transfected cells expressing the activated kinase are then exposed to the putative inhibitory agents, advantageously in the absence of external activating agents. The activity of the inhibitory agents may be assessed by any of the methods referred to above.

The invention further includes a method for identifying a kinase enzyme which phosphorylates the T₂₂₉ activating site of p70^{S6K} comprising screening cell extract fractions for an activity capable of activating a T₃₈₉->E, D₃E mutant of p70^{S6K}, and subsequently purifying the kinase from an activity-containing fraction.

The kinase may be purified from the fraction by established methodology. Suitable purification methods include chromatography in general, based on separation by charge difference and/or size difference, such as ion exchange chromatography using an exchange group such as DEAE or CM bound to a solid phase packing material such as cellulose, dextran, agarose or polystyrene. Other methods include hydroxyapatite column chromatography (see, for example, Gorbunoff, (1985) Methods in Enzymology, 117, 370-380), and general affinity chromatography using glass beads or reactive dyes as affinity agents. A general guide to protein purification is given in Deutscher (1990) Guide to Protein Purification, Academic Press, Inc., San Diego, Calif., USA, 317-328.

The same T₃₈₉->E, D₃E mutant may then be used to identify and isolate a kinase enzyme responsible for activating T₃₈₉ of p70^{S6K} and regulating the activity of p70^{S6K}. In order to isolate this kinase, cell extract fractions supplemented with T₂₂₉ activating kinase are screened for an activity capable of activating the mutant, which remains sensitive to activation. Alternatively, wild-type p70^{S6K} may be used in a similar experiment.

Purification of the T₃₈₉ kinase may be accomplished as described above.

The invention is further described below, for the purpose of illustration only, in the following examples.

### EXAMPLE 1

Identification of Rapamycin Sensitive Phosphorylation Sites

Unlike the identification of the initial S/TP phosphorylation sites residing in the autoinhibitory domain (referred to herein as D₃E), attempts to identify the rapamycin sensitive sites S₄₀₄, T₂₂₉ and T₃₈₉, employing a number of proteases are ineffective, as the phosphopeptides containing these sites do not migrate with predicted mobilities (Boyle et al, (1991) Methods Enzymol. 201, 110-149) of proteolytic peptides derived from p70^{S6K}. Therefore, picomolar amounts of these peptides are produced for direct analysis from a mutant p70^{S6K} construct in which acidic residues are substituted at the four known S/TP sites of phosphorylation, termed herein p70^{S6K}D₃E. A His-tagged p70^{S6K}D₃E mutant is obtained by introducing 6 His codons after the initiator Met of p70^{S6K}D₃E. The insertion is performed by polymerase chain reaction. Resulting mutants are inserted into a human CMV promoter-driven expression vector (Ferrari et al., 1993). Following transient transfection into human 293 cells, the ³²P-labelled rapamycin sensitive tryptic phosphopeptides b, c and d are purified from the mutant kinase as follows: extracts are centrifuged at 18000×g for 20 min at 4° C., the pellet discarded and the His-p70^{S6K}D₃E purified by sequential chromatography on cation exchange and nickel chelating columns followed by preparative SDS-PAGE. The purified protein is electroeluted, precipitated by the addition of trichloroacetic acid to 15% (w/v), performic acid oxidised on ice for 1 hr, lyophilised and digested with trypsin in 100 mM NH₄ HCO₃ pH 8.3. Digestion of ˜1 mg of purified kinase is carried out at 37° C. for 36 h with 3 additions of 5 μg of TPCK-treated trypsin (Worthington) every 12 h. The resulting peptides are separated by reversed phase chromatography on a glass lined C18 column (2×100 mm, SGE) and detected using a PhosphorImager (Molecular Dynamics) following either TLC, in the case of phosphopeptides c and d, or TLE at pH 1.9, in the case of phosphopeptide b. Phosphopeptides b-d are then purified to homogeneity by preparative 2-D TLE/TLC on cellulose plates. Following autoradiography, peptides are scraped from the plates and eluted from the cellulose matrix with 30% (v/v) acetonitrile.

All three of the purified phosphopeptides isolated from the overexpressed p70^{S6K}D₃E migrate in identical position as their respective counterparts from endogenous p70^{S6K} derived from serum-stimulated Swiss 3T3 cells. Phosphoamino acid analysis demonstrates that phosphopeptide b contains phosphoserine whereas c and d contain phosphothreonine.

Chemical sequencing, mass spectrometry and phosphate release identify phosphopeptide c as E219-Y236 being singly phosphorylated at T₂₂₉. Similar analysis identifies peptides b and d as E401-K408 and G387-K400, also singly phosphorylated at either S₄₀₄ or T₃₈₉, respectively. All three phosphopeptides are a typical tryptic cleavage products, explaining the initial failure of the mapping program to predict peptides matching their mobilities (Boyle et al., 1991).

### EXAMPLE 2

Determination of the Primary Target of Rapamycin Action.

In contrast to the S/SP sites in the autoinhibitory domain, T₂₂₉ is found in the highly conserved “T loop” of the catalytic domain, whereas T₃₈₉ and S₄₀₄ reside in a linker region between the catalytic and autoinhibitory domains. Furthermore, all three residues are flanked by large aromatic residues. In a number of mitogen-activated kinases, increased phosphorylation at sites in the “T loop” is essential for kinase activity. This fact suggests that the primary target of p70^{S6K} inactivation by rapamycin is T₂₂₉. To examine this possibility, p70^{S6K} activity is compared to the phosphorylation state of the enzyme in serum-stimulated Swiss 3T3 cells at times following rapamycin treatment, wherein either the vehicle (5 μl of 0.1% ethanol per 10 ml medium) or 20 nM rapamycin (5 μl of 4 μM rapamycin per 10 ml medium) is added to cells after serum stimulation. In order to analyse kinase activity, total protein extract from Swiss 3T3 cells (20 μg) is diluted in extraction buffer to final volume of 200 μl followed by immunoprecipitation with addition of 5 μl M5 antibody (\check{S}u\check{s}a et al., 1992). Kinase activity is assayed as described previously (Lane and Thomas, 1991).

Within 4 to 5 min of the addition of 5 nM rapamycin, 50% of p70^{S6K} activity is abolished with kinase activity reaching basal levels within 15 to 30 min. This loss in kinase activity is reflected as a progressive increase in the mobility of p70^{S6K} on SDS-PAGE. The activated form of p70^{S6K} is represented by two electrophoretic species, with the majority of the protein migrating with the slower form. Following rapamycin treatment, the mobility of these two species increases, with loss of activity most closely paralleling loss of the slowest migrating species. Though no p70^{S6K} activity can be detected 30 min post rapamycin treatment, a slower migrating species of the kinase indicates that the kinase is still phosphorylated.

In order to correlate loss of kinase activity with phosphorylation state, phosphopeptides were prepared as described above and mapped as described previously (Ferrari et al., 1992) with the following modifications; the electroeluted protein is precipitated with 15% w/v trichloroacetic acid and performic acid oxidised prior to digestion with trypsin (3×5 μg over 24 h); following separation of the phosphopeptides on thin layer plates, the resultant chromatograms are visualised using a PhosphorImager and ImageQuant Software (Molecular Dynamics). Surprisingly, two-dimensional phosphopeptide mapping of p70^{S6K} isolated at selected time points during rapamycin-induced inactivation reveals that kinase activity decreases more rapidly than phosphate is lost from T₂₂₉. Indeed, p70^{S6K} inactivation more closely parallels loss of phosphate from T₃₈₉ and S₄₀₄. The first phosphorylation site to disappear is T₃₈₉, which is strongly reduced within 4 min of rapamycin treatment, is barely detectable after 15 min, and no longer visible after 30 min of rapamycin treatment. A similar pattern, though slightly delayed in time, is also observed for S₄₀₄. Thus, it appears that rapamycin inactivates p70^{S6K} by inducing dephosphorylation of sites located outside the catalytic domain.

### EXAMPLE 3

Mutation of Phosphorylated Sites

In an attempt to assess their relative importance in mitogen-induced p70^{S6K} activation, each of the rapamycin sensitive phosphorylation sites is mutated to either a neutral or acidic residue (Ferrari et al., 1993) using site-directed mutagenesis of the rapamycin sensitive sites, carried out on the myc-epitope tagged p70^{S6K} and p70^{S6K}D₃E clones using the Altered Site II Mutagenesis System (Promega). The mutant constructs are transiently transfected into human 293 cells (10⁶ per 10 cm plate), and the cells quiesced by serum deprivation for 24 hrs and labelled with ³²Pi (1 μCi/5 ml) for 8 hours before further stimulation with 10% serum for 60 min. Mutation of T₂₂₉, to either alanine or glutamic acid, abolished kinase activity, suggesting a key role for this site, but prohibiting further assessment of the importance of phosphorylation at this position. In contrast to T₂₂₉, substitution of either an alanine or an aspartic acid for S₄₀₄ had no effect on the extent of p70^{S6K} activation. That the alanine substitution is as effective as the aspartic acid mutation suggests that this phosphorylation site is not the principal target of rapamycin action. In parallel experiments, the basal activity of the E₃₈₉ mutant in quiescent cells is found to be 3-fold higher than wild type p70^{S6K}, but its ability to respond to serum is strongly diminished, to approximately 50% of the wild type construct. In contrast, no activity can be detected in the A₃₈₉ mutant from either quiescent or serum-stimulated cells. The location of T₃₈₉ suggests that it may function with the autoinhibitory domain in regulating p70^{S6K} activity. This, combined with the fact that p70^{S6K}D₃E has higher basal kinase activity than p70^{S6K}, suggests that the activity of the E₃₈₉ mutant may be augmented if placed in a p70^{S6K}D₃E background. This is found to be the case, with the basal activity of this mutant being higher than that of either p70^{S6K}D₃E or p70^{S6K}-E₃₈₉ and the serum-stimulated level of activity being equal to that of the wild type construct. The results indicate that phosphorylation of T₃₈₉ plays a critical role in regulating kinase activity. Interestingly, the motif surrounding T₃₈₉, though not previously noted, is conserved in many members of the second messenger family of protein kinases. In the case of PKCα, this domain has been implicated as playing a critical role in the maintenance of kinase activity, supporting a role in the regulation of p70^{S6K} activity.

In order to assess the resistance of the E₃₈₉ mutant to rapamycin inactivation, serum-stimulated cells transiently expressing p70^{S6K} or p70^{S6K}D₃E, as well as each construct containing the E₃₈₉ mutation, are treated with 20 nM rapamycin for 15 min. The results show that the two parent constructs rapidly lose activity whereas the E₃₈₉ mutants are 50-70% resistant to rapamycin-induced inactivation. That the two constructs harbouring the E₃₈₉ mutation still exhibits some rapamycin sensitivity, suggested a secondary target of rapamycin-induced p70^{S6K} inactivation. Indeed, preliminary data suggests that loss of activity in the E₃₈₉ mutants correlates with dephosphorylation of T₂₂₉. Unexpectedly, however, longer treatment with rapamycin shows that after 1 h, the p70^{S6K}D₃E E₃₈₉ mutant is still 35-40% resistant, a level of activity which does not decrease after a further 2 h treatment with rapamycin. Under identical conditions, the p70^{S6K}D₃E parent construct returns to basal levels within 15 min. Similar results are obtained with p70^{S6K}-E38₉, though the extent of resistance is lower, approximately 21%. The results from the mutational analysis, together with the time course of site specific dephosphorylation following rapamycin treatment, strongly supports T₃₈₉ as being the principal target of rapamycin-induced p70^{S6K} inactivation.

1. A protein kinase having a structure related to that of a kinase enzyme of the p70^{S6K} signalling pathway wherein a serine or threonine residue homologous to T₃₈₉ in p70^{S6K} is replaced with an acidic amino acid residue in order to confer constitutive activity.

2. A protein kinase according to claim 1 which is selected from the group consisting of: p70^{S6K}, PKCα, PKCγ, PKCδ, PKCε, RSK xen1, RSK mα1, RAC-PK/Akt α, RAC-PK/Akt β, KPC3 drome, KPC2 caeel, ATPK1, PK2 dicdi, SCH9 and YPK1.

3. A protein kinase according to claim 1 or claim 2 comprising the additional replacement of serine or threonine residues with acidic amino acid residues a at a position homologous to a position in p70^{S6K} selected from the group consisting of S₄₁₁, S₄₁₈, S₄₂₄, T₄₂₁ and S₄₀₄.

4. A protein kinase according to claim 3 wherein S₄₁₁, S₄₁₈, S₄₂₄ and T₄₂₁ have been mutated to acidic amino acid residues.

5. A protein kinase according to claim 1 wherein the residue homologous to T₃₈₉ of p70^{S6K} is replaced with glutamic acid.

6. A protein kinase according to any preceding claim, wherein threonine residues are replaced with glutamic acid and serine residues are replaced with aspartic acid.

7. A method for screening compounds for activity as inhibitors of a protein kinase related to a kinase enzyme of the p70^{S6K} signalling pathway comprising assaying for dephosphorylation of the T₃₈₉ homologous site in said protein kinase in response to the compounds.

8. A method according to claim 7 wherein dephosphorylation of the T₃₈₉ homologous site is assayed by two dimensional tryptic phosphopeptide mapping.

9. A method for screening compounds for activity as inhibitors of a kinase enzyme of the p70^{S6K} signalling pathway comprising the steps of:
(a) combining a constitutively active kinase according to any one of claims 1 to 6 with a direct or indirect substrate therefor;
(b) exposing the combination to a candidate inhibitory agent; and
(c) assessing the phosphorylation state of the substrate, lack of phosphorylation being indicative of inhibition of the kinase by the agent.

10. A dominant negative mutant of a protein kinase having a structure related to that of a kinase enzyme of the p70^{S6K} signalling pathway wherein T₂₂₉ is mutated to Alanine.

11. A method for identifying a kinase enzyme capable of activating a p70^{S6K}-related kinase, comprising assaying a compound or mixture of compounds for ability to activate a mutant of a p70^{S6K}-related kinase.
